(19) United States
(12) Patent Application Publication
(10) Pub. No.: US 2007/0010008 A1
(43) Pub. Date: Jan. 11, 2007

(54) Ex vivo expansion of primary animal cells

(75) Inventors: Scheffer C.G. Tseng, Pinecrest, FL (US); Hua He, Miami, FL (US); Wei Li, Miami, FL (US)

Correspondence Address: WILSON SONSINI GOODRICH & ROSATI, 650 PAGE MILL ROAD, PALO ALTO, CA, 94304-1050, US

(73) Assignee: TissueTech, Inc.

(21) Appl. No.: 11/476,376

(22) Filed: Jun. 28, 2006

Related U.S. Application Data
(60) Provisional application No. 60/695,051, filed on Jun. 29, 2005.
(60) Provisional application No. 60/695,576, filed on Jun. 30, 2005.
(60) Provisional application No. 60/703,188, filed on Jul. 28, 2005.

(51) Int. Cl. C12N 5/06; C12N 5/08

(52) U.S. Cl. 435/325; 435/366

(57) ABSTRACT

An ex vivo method of expanding animal cells whose differentiation state is controllable by modulating TGF-β signaling includes the steps of: (a) providing an animal subject having cells with a first phenotype; (b) isolating the cells from the animal subject; (c) placing the cells in an ex vivo culture system including a culture vessel having at least one surface and a medium in contact with the at least one surface, the medium being essentially free of intact amniotic membrane and feeder cells; and (d) culturing the cells in the medium under conditions which downregulate TGF-β signaling in the cells and allow the cells to proliferate while maintaining the first phenotype.

### CROSS-REFERENCE TO RELATED APPLICATION

The present application claims the priority of U.S. provisional application No. 60/695,051 filed Jun. 29, 2005; 60/695,576 filed Jun. 30, 2005; and 60/703,188 filed Jul. 28, 2005.

### STATEMENT AS TO FEDERALLY SPONSORED RESEARCH

Certain aspects of the invention were made with United States government support under grant number RO1 EY06819 awarded by the National Institutes of Health. The United States government may have certain rights in the invention.

### FIELD OF THE INVENTION

The invention relates generally to the fields of developmental biology and tissue culture. More particularly, the invention relates to methods and systems for expanding animal cells in ex vivo cultures while preventing their differentiation through conditions or agents which downregulate TGF-β signaling in the cells.

### BACKGROUND

Cellular or cell-based therapy is the replacement of unhealthy, damaged, or diseased cells or tissues with new ones. Blood transfusions and bone marrow transplantation are prime examples of the successful application of cell-based therapeutics, but recent advances in cellular and molecular biology have expanded the potential applications of this approach to a wide variety of clinical disorders. The realization of these applications, however, depends on obtaining or culturing the cell type of interest in sufficient numbers for transplantation into the damaged or diseased tissue or organ. Theoretically, cells of interest can be explanted from an animal or human subject and introduced into a primary cell culture system for expansion. In practice, however, defining and refining the conditions that allow primary cell expansion without phenotypic changes (e.g., differentiation from an epithelial cell phenotype to a fibroblastic phenotype) has required a prodigious effort. For culturing epithelial cells, one common method involves the use of murine fibroblast feeder layers. A drawback of this method, however, is the inclusion of a murine cell, which potentially can transmit xenogenic diseases to the cultured cells. A common method for culturing limbal epithelial cells and corneal stromal keratocytes involves seeding the cells on a sheet of intact amniotic membrane (AM) (see e.g., He et al., Invest Ophthalmol Vis Sci. 47:151-157, 2006). By seeding the cells on such sheets, however, further manipulation of the ex vivo expanded cells is restricted.

### SUMMARY

The invention is based on the elucidation of particular cell culture conditions that allow expansion of explanted primary cells without causing their differentiation to a different phenotype. As described below, in one embodiment of the invention, it was discovered that TGF-β signaling plays a critical role in regulating the differentiation of a variety of cells including stem cells such as limbal epithelial progenitor cells and differentiated cells such as corneal keratocytes. Conditions that downregulate TGF-β signaling to promote cell expansion without cell differentiation include culturing the cells at low density in a low Ca²⁺ and serum-free medium. Agents that downregulate TGF-β signaling to promote cell expansion without cell differentiation include isolated AM; AM stromal matrix; processed AM; AM extracts (AME); components derived from AM such as hyaluronic acid (HA), HA-inter-α-trypsin-inhibitor heavy chain (HA-ITI), lumican; TSG-6; Pentraxin (PTX3); Thrombospondin; anti-TGF-β antibodies; and inhibitors of components in the TFG-β signaling pathway such as serine/threonine kinase inhibitors and Smad components. In some embodiments, ex vivo cell culture systems include a cell culture vessel that houses a cell to be expanded and a medium that has a low [Ca²⁺] and is serum-free (and thus TGF-β free) for supporting expansion of the cell. In other embodiments, ex vivo cell culture systems include a cell culture vessel that houses a cell to be expanded and a medium containing serum (and thus TGF-β) and a high [Ca²⁺] (between about 0.1 mM and 1.8 mM) and an agent or condition that downregulates TGF-β signaling. Because serum (e.g., fetal bovine serum (FBS)) contains TGF-β it normally promotes cellular differentiation. Both high [Ca²⁺] and FBS are known to upregulate TGF-β signaling. However, agents that downregulate TGF-β signaling described herein mitigate this effect and prevent differentiation of the cells.

The invention thus provides a means for culturing and expanding explanted cells important in maintaining tissue integrity in long term ex vivo cultures without undesired differentiation. Accordingly, both stem cells and differentiated cells can be grown to numbers sufficient for use in cell-replacement or tissue-engineering therapies for treating various pathologies such as cancer and HIV-associated pathologies. The invention further provides a means of promoting the differentiation of an expanded cell by contacting the cell with TGF-β (or a suitable agonist thereof) or by removing an agent/condition that downregulates TGF-β signaling.

Accordingly, the invention features an ex vivo method of expanding animal cells whose differentiation state is controllable by modulating TGF-β (e.g., TGF-β isoforms 1 and 2) signaling. The method includes the steps of: (a) providing an animal subject including cells having a first phenotype; (b) isolating the cells from the animal subject; (c) placing the cells in an ex vivo culture system including a culture vessel having at least one surface and a medium in contact with the at least one surface, the medium being essentially free of intact AM and feeder cells; and (d) culturing the cells in the medium under conditions which downregulate TGF-β signaling in the cells and allow the cells to proliferate while maintaining the first phenotype. In one aspect of this method, the cells are differentiated cells such as keratocytes. In another aspect of this method, the cells are undifferentiated such as stem cells, including for example, limbal epithelial progenitor cells, umbilical cord epithelial cells and amniotic membrane epithelial cells.

In the method, the conditions which downregulate TGF-β signaling in the cells can include culturing the cells in a serum-free medium lacking TGF-β and having less than about 0.1 mM Ca²⁺ (e.g., KSFM). In another example of a method, the conditions which downregulate TGF-β signaling in the cells can include culturing the cells in a medium containing serum, TGF-β and Ca²⁺ (as high as about 1.8 mM Ca²⁺, e.g., DMEM or SHEM).

The conditions which downregulate TGF-β signaling in the cells can include culturing the cells in a serum-containing medium and contacting the cells with an agent that downregulates TGF-β signaling in the cells. Agents that can be used to downregulate TGF-β signaling cells include those that downregulate transcription of a TGF-β gene in the cells (e.g., an anti-sense nucleic acid or an siRNA), those that specifically bind TGF-β, antibodies (e.g., those that specifically bind TGF-β or a receptor for TGF-β), those that antagonize a receptor for TGF-β (e.g., an antibody, a modified form of TGF-β, and cystatin C), those that inhibit protein phosphorylation (e.g., a serine/threonine protein kinase inhibitor), and those that prevent translocation of a Smad protein from the cytoplasm of the cell to its nucleus. AM-based agents that can be used to downregulate TGF-β signaling in cells at the transcriptional level include extracts of AM, a purified component of AM such as purified hyaluronic acid (HA), HA-ITI, lumican and combinations thereof. Additional inhibitors of TGF-β signaling include TSG-6, pentraxin, and thrombospondin.

In another aspect the invention features an ex vivo cell culture system including a vessel including animal cells whose differentiation state is controllable by modulating TGF-β signaling, wherein the animal cells have been expanded by culturing in a medium free of intact AM under conditions which downregulate TGF-β signaling in the cells to allow the cells to proliferate without changes to their phenotype.

Also within the invention is an ex vivo method of preferentially expanding limbal epithelial progenitor cells in a cell culture initiated with a mixture of limbal progenitor cells and transient amplifying cells (TACs). TACs secrete TGF-β1 and TGF-β2 as negative factors that affect limbal progenitor cells. This method includes the steps of: (a) placing the mixture of limbal epithelial progenitor cells and TACs in an ex vivo culture system including a culture vessel having at least one surface and a medium in contact with the at least one surface, wherein the mixture of cells is seeded in the culture system at a cell density (e.g., between about 10 and 500 cells/cm²) sufficiently low to prevent the TACs from having a negative paracrine effect in the limbal epithelial progenitor cells and (b) culturing the cells in the in ex vivo culture system for a time period exceeding the lifespan of the TACs (e.g., greater than about 3 weeks) under conditions suitable for expanding the limbal epithelial progenitor cells. As used herein, the phrase “stem cell” means an undifferentiated cell that retains the ability to divide and differentiate into other cell types. A stem cell can be totipotent, pluripotent, or multipotent.

By the phrase “differentiated cell” is meant a cell that is more differentiated than the stem cell from which it originated. An example of a differentiated cell is a keratocyte, which expresses cellular markers not expressed by the stem cells from which the keratocytes originated.

When referring to a cell culture system “essentially free of” a substance (e.g., intact AM or feeder cells) is meant that that substance is not present in a sufficient amount to exert a detectable effect on the cells in the culture system (e.g., to cause or prevent a phenotypic change in the cells).

As used herein, an “antibody” is an intact immunoglobulin or an antigen-binding fragment or derivative thereof.

When referring to a protein or other biological molecule, “purified” means separated from components that naturally accompany such molecules. Typically, a molecule is purified when it is at least 30% (e.g., 40%, 50%, 60%, 70%, 80%, 90%, and 100%), by weight, free from the proteins or other naturally-occurring organic molecules with which it is naturally associated. Purity can be measured by any appropriate method, e.g., column chromatography, polyacrylamide gel electrophoresis, or HPLC analysis.

By “bind”, “binds”, or “reacts with” is meant that one molecule recognizes and adheres to a particular second molecule in a sample, but does not substantially recognize or adhere to other molecules in the sample. Generally, a first molecule which “specifically binds” a second molecule has a binding affinity greater than about 105 to 106 liters/mole for the second molecule.

Unless otherwise defined, all technical and legal terms used herein have the same meaning as commonly understood by one of ordinary skill in the art to which this invention belongs. Although methods and materials similar or equivalent to those described herein can be used in the practice or testing of the present invention, suitable methods and materials are described below. All publications mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention encompasses systems and methods for expanding primary animal cells without causing changes to their phenotype. The systems and methods of the invention involve manipulating TGF-β signaling to control the progression of cells from one phenotype to another. In particular, TGF-β signaling is downregulated to prevent the differentiation of a cell during expansion, while restoring normal TGF-β signaling by removing a composition and/or condition that downregulates TGF-β signaling can be used to promote the differentiation of the cell.

The below described preferred embodiments illustrate adaptation of these compositions and methods. Nonetheless, from the description of these embodiments, other aspects of the invention can be made and/or practiced based on the description provided below.

### Biological Methods

Methods involving conventional biological techniques are described herein. Such techniques are generally known in the art and are described in detail in methodology treatises such as Molecular Cloning: A Laboratory Manual, 3^{rd} ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 2003 (with periodic updates). Various conventional techniques for culturing animal cells are described in Culture of Animal Cells: A Manual of Basic Technique, 4^{th} ed., R. Ian Freshney, Wiley-Liss, Hoboken, N.J., 2000, and Animal Cell Culture Techniques (Springer Lab Manual), M. Clynos, Springer-Verlag, New York, N.Y., 1998. Methods involving protein analysis and purification are also known in the art and are described in Protein Analysis and Purification: Benchtop Techniques, 2^{nd} ed., Ian M. Rosenberg, Birkhauser, New York, N.Y., 2004.

### Ex Vivo Method of Expanding Animal Cells Whose Differentiation State is Controllable by Modulating TGF-β Signaling

The invention includes methods of expanding animal cells ex vivo whose differentiation state is controllable by modulating TGF-β signaling. Such a method includes several steps. The first step includes providing an animal subject having cells of a first phenotype followed by isolating the cells from the animal subject. The next step includes placing the cells in an ex vivo culture system including a culture vessel having at least one surface and a medium in contact with the at least one surface, the medium being essentially free of intact AM and feeder cells. Lastly, the method includes culturing the cells in the medium under conditions which downregulate TGF-β signaling in the cells to allow the cells to proliferate while maintaining the first phenotype.

As described herein, a cell having a first phenotype can be any cell type in which TGF-β signaling modulation affects its differentiation state. Such cells might be stem cells or differentiated cells. Examples of stem cells include totipotent stem cells, pluripotent stem cells, and multipotent stem cells. A number of adult, embryonic, and cord blood stem cells are known, including hematopoietic stem cells, pancreatic stem cells, mesenchymal stem cells, bone marrow stromal stem cells, adipose derived adult stem cells, olfactory stem cells, gastrointestinal stem cells, mammary gland stem cells, and limbal epithelial progenitor cells. Differentiated cells might include epithelial cells, fibroblasts, myocytes, pancreatic β cells, blood cells, neurons, smooth muscle cells, fat cells, oligodendrocytes, alveolar cells, epidermal cells, and keratocytes.

To isolate cells from an animal subject, any suitable method may be used. As one example, a typical method of isolating keratocytes from an animal subject includes removing an anterior corneoscleral segment from the globe of the animal subject's eye by cutting near the limbus with Wescott's scissors or other appropriate cutting implement (see Kawakita et al., Invest Ophthalmol. Vis. Sci. 47:1918-1927, 2006 and Espana et al., Invest Ophthalmol. Vis. Sci. 46:4528-4535, 2005). A central cornea can be obtained with an 8.0 mm Hessburg-Barron trephine or other suitable trephine system and transferred to an appropriate medium (e.g., KSFM). After removing Descemet's membrane and the corneal epithelium by digestion with an appropriate protease (e.g., Dispase II for 16 h at 4° C.), the remaining corneal stroma is incubated at 37° C. for a suitable amount of time (e.g., 16 h) in medium (e.g., DMEM) containing collagenase and any other appropriate components for digestion (e.g., HEPES, gentamicin, amphotericin) on a suitable culture substrate or vessel (e.g., multi-well plate, plastic dish). Then, cells are resuspended in a suitable medium (e.g., KSFM), centrifuged to remove residual matrices, resuspended again, and seeded on an appropriate culture substrate or vessel (e.g., multi-well plate, plastic dish) in a suitable medium such as KSFM or DMEM containing ITS or 10% FBS. When this primary culture reaches approximately 80% confluence, cells are rendered into single cells by incubation in an appropriate solution (e.g., balanced salt solution (BSS) containing 0.25% trypsin/lmM EDTA) at 37° C. for approximately 1 to 5 minutes, and the enzymatic reaction is stopped by adding soybean-trypsin inhibitor. After centrifuging (e.g., at 800×g for 5 minutes), the cells are resuspended in a suitable medium (e.g., KSFM) and cultured until use.

A typical method of isolating limbal epithelial progenitor cells includes first isolating a corneoscleral ring from a cornea (as described in He et al., Invest. Ophthalmol. Vis. Sci 47:151-157, 2005; Kawakita et al., Am. J. Pathol. 167:381-393, 2005). Then, limbal corneal epithelial sheets are isolated from the corneoscleral ring by digestion with a suitable protease (e.g., 10 mg/ml Dispase II in KSFM at 37° C. for 2 hours). Alternatively, the limbal corneal epithelial cells can be isolated from the corneoscleral ring by treatment with cell dissociation buffer prior to culturing in an appropriate medium (e.g., SHEM and KSFM+S with or without 3T3 cells). The sheets are trypsinized and cultured on a suitable culture substrate or vessel (e.g., multi-well plate or plastic with or without 3T3 fibroblast feeder layers) in an appropriate medium (e.g., SHEM).

In another method of isolating limbal epithelial progenitor cells, excessive sclera, the iris, the corneal endothelium, the conjunctiva, and Tenon's capsule are removed from a donor eye (e.g., human donor eye). Then, the limbal ring is separated by a 7.5 mm trephine or other suitable trephine system from the cornea. Each limbal ring is washed with an appropriate medium (e.g., rinsed 3 times with SHEM media) and is then exposed to a suitable protease (e.g., 1.2 units/ml Dispase II for 10 min) in a suitable medium (e.g., Mg²⁺ and Ca²⁺-free HBSS) under suitable conditions (e.g., at 37° C. under 95% humidity and 5% CO₂).

Methods of expanding animal cells ex vivo whose differentiation state is controllable by modulating TGF-b signaling as described herein include placing the cells in an ex vivo culture system including a culture vessel having at least one surface and a medium in contact with the at least one surface, the medium being essentially free of AM stromal matrix and feeder cells. In ex vivo culture systems of the invention, any suitable vessel can be used. Examples of suitable vessels include traditional tissue culture substrates such as 6-, 24-, and 96-well plates, Petri dishes, flasks, bottles, plastic, and coverslips.

Culture systems involving the use of feeder layers and intact AM are generally undesirable because feeder layers have been shown to transmit xenogenic diseases to the cells being cultured and use of intact AM restricts further manipulation of the expanded cells. Thus, examples of suitable media for use in ex vivo culture systems include a medium essentially free of intact AM and feeder cells. Typically, any culture media that enable the expansion of stem cells while maintaining the “stemness” or stem cell qualities of the stem cells, and differentiated cells without the differentiated cells turning to another cell type, are particularly useful. In some embodiments, a culture medium that inhibits TGF-β signaling in the cells is preferred. To downregulate TGF-β signaling in the cells, the cells are typically cultured in a serum-free medium having a low [Ca²⁺] and no added TGF-β (e.g., KSFM). For example, a medium having less than about 10 ng/ml (e.g., less than 11, 10, 9, 8, 7, 6, 5, 4, 3, 2, 1, 0.5, 0.25, 0.1, 0.05, and 0.01 ng/ml) of TGF-β and less than about 0.1 mM (e.g., less than 0.11, 0.10, 0.09, 0.08, 0.07, 0.06, 0.05, 0.04, 0.03, 0.02, 0.01, and 0.005 mM) Ca²⁺ can be used. For culturing keratocytes, a typical medium is KSFM (cat. no. 17005-042, Gibco, Carlsbad, Calif.), a defined keratocyte serum-free medium that has a lower [Ca²⁺] (e.g., less than 0.10 mM such as between about 0.03 and 0.09 mM) than DMEM and that has no FBS (serum). For expanding limbal epithelial progenitor cells, the cells can also be cultured in (a) KSFM, (b) seeded on AM and cultured in KSFM, and (c) cultured in KSFM to which AME has been added.

In some methods and ex vivo culture systems in which TGF-β signaling is downregulated in the cells, the medium contains serum (and thus TGF-β) and a [Ca²⁺] greater than about 0.1 mM (e.g., 0.12, 0.15, 0.2, 0.3, 0.4, 0.5, 0.6, 0.7, 0.8, 0.9, 1.0, 1.2, 1.4, 1.6, 1.8, 2.0 mM Ca²⁺) and the conditions which downregulate TGF-β signaling in the cells include contacting the cells with an agent that downregulates TGF-β signaling in the cells (e.g., AME and AM components). For example, instead of using KSFM for expanding animal cells, an alternative media such as SHEM (described in Meller et al., Br J Ophthalmol., 86:463-471, 2002; Grueterich and Tseng, Arch Ophthalmol., 120:783-790, 2002) can be used. If SHEM is used, however, an agent that downregulates TGF-β signaling in the cells (e.g., AME, AM components) is added to the media because SHEM contains FBS and a high [Ca²⁺]. In other embodiments, conditions which downregulate TGF-β signaling in the cells include seeding the cells at a cell density sufficiently low to prevent the TACs from having a negative paracrine effect (e.g., secretion of TGF-β1 or TGFβ2) on the limbal epithelial progenitor cells and for a time period that is greater than that of the TACs. In such embodiments, the cell density is typically between about 10 and 500 cells/cm² and the time period is greater than about 3 weeks.

In the ex vivo culture systems and methods described herein, any suitable agent for downregulating TGF-β signaling in a cell can be used. Examples of agents that downregulate TGF-β signaling include those that downregulate transcription of TGF-β gene in the cells. In some cases, the agent may specifically bind to TGF-β (e.g., an antibody), while in other cases, the agent may antagonize a receptor for TGF-β. Small molecule TGF-β signaling inhibitors such as SB-431542 (Hjelmand et al., Mol Cancer Ther. 3(6):737-745, 2004) and those described below might be used to downregulate TGF-β signaling in cells. A serine/threonine protein kinase inhibitor, a molecule that prevents translocation of a Smad protein from the cytoplasm of the cell to its nucleus, TSG-6, Pentraxin, Thrombospondin, AME, processed non-intact AM, and a purified component of AM (e.g., HA, HA-ITI, lumican) are further examples of agents that can be used to downregulate TGF-β signaling in the cells at the transcriptional level. In some experiments described herein, limbal epithelial progenitor cells and keratocytes cultured in medium containing AME were expanded while maintaining their characteristic phenotypes.

A suitable form of isolated AM is described in U.S. Pat. Nos. 6,152,142, and 6,326,019. Processed AM might take the form of a powder (e.g., lyophilized and ground or pulverized AM) or other suitable form of AM. In addition, portions of AM might be used such as extracts of AM (see, e.g., U.S. provisional patent application 60/657,399) or purified components of AM such as extracellular matrix components such as HA, HA-ITI, and lumican. Methods of culturing cells on AM (e.g., AM stromal matrix) in culture medium containing serum that prevent the differentiation of the cells are described herein.

A number of additional agents that downregulate TGF-β signaling are known and can be used in ex vivo culture systems and methods described herein. Typical agents for modulating expression (and thus signaling) of intracellular proteins are mutants proteins, nucleic acids, and small organic or inorganic molecules. Examples of proteins that can modulate TGF-β expression and/or activity in a cell include variants or native TGF-β proteins or receptors thereof that can compete with a native TGF-β protein or receptor thereof. Such protein variants can be generated through various techniques known in the art. For example, protein variants can be made by mutagenesis, such as by introducing discrete point mutation(s), or by truncation. Mutation can give rise to a protein variant having substantially the same, or merely a subset of the functional activity of a native protein.

Another agent that can modulate TGF-β signaling is a TGF-β-based or TGF-β receptor-based non-peptide mimetic or chemically modified form of a TGF-β or a TGF-β receptor that disrupts binding of between a TGF-β protein and its receptor. See, e.g., Freidinger et al. in Peptides: Chemistry and Biology, G. R. Marshall ed., ESCOM Publisher: Leiden, Netherlands, 1988). TGF-β proteins or receptors thereof may, for example, be chemically modified to create protein derivatives by forming covalent or aggregate conjugates with other chemical moieties, such as glycosyl groups, lipids, phosphate, acetyl groups and the like. Covalent derivatives of a protein can be prepared by linking the chemical moieties to functional groups on amino acid side chains of the protein or at the N-terminus or at the C-terminus of the polypeptide.

The agent that directly reduces TGF-β signaling can also be a nucleic acid that modulates expression of a TGF-β protein or receptor thereof. For example, the nucleic acid can be an antisense nucleic acid that hybridizes to mRNA encoding the TGF-β or receptor thereof. Antisense nucleic acid molecules for use within the invention are those that specifically hybridize (e.g. bind) under cellular conditions to cellular mRNA and/or genomic DNA encoding the protein of interest in a manner that inhibits expression of the protein, e.g., by inhibiting transcription and/or translation. Antisense constructs can be delivered using an expression vector plasmid or any other suitable means.

Ribozyme molecules designed to catalytically cleave TGF-β or TGF-β receptor mRNA transcripts can also be used to prevent translation of and expression of these proteins (see, e.g., PCT Publication No. WO 90/11364, published Oct. 4, 1990; Sarver et al., Science 247:1222-1225, 1990 and U.S. Pat. No. 5,093,246). In other embodiments, endogenous TGF-β or TGF-β receptor gene expression might be reduced by targeting deoxyribonucleotide sequences complementary to the regulatory region of the TGF-β or TGF-β receptor gene (i.e., the TGF-β or TGF-β receptor promoter) to form triple helical structures that prevent transcription of the targeted gene. (See generally, Helene, C., Anticancer Drug Des. 6(6):569-84, 1991; Helene, C., et al., Ann. N.Y. Acad. Sci. 660:27-36, 1992; and Maher, L. J., Bioassays 14(12):807-15, 1992). Inhibition of TGF-β gene expression might also be performed using RNA interference (RNAi) techniques. Such techniques are described in, for example, Zhou et al., Curr Top Med Chem, 6:901-911, 2006; Morris, K. V., BioTechniques April, Suppl:7-13, 2006; and Gilmore et al., Curr Drug Deliv. 3:147-150, 2006.

An example of a protein that can modulate TGF-β signaling is an antibody that specifically binds a TGF-β or a TGF-β receptor. Such an antibody can be used to interfere with the interaction of the TGF-β and its receptor or to directly antagonize the receptor.

### Ex Vivo Method of Preferentially Expanding Limbal Epithelial Progenitor Cells

An ex vivo method of preferentially expanding limbal epithelial progenitor cells in a cell culture initiated with a mixture of limbal progenitor cells and TACs includes the steps of placing the mixture of limbal epithelial progenitor cells and TACs in an ex vivo culture system including a culture vessel having at least one surface and a medium in contact with the at least one surface, and culturing the cells in the ex vivo culture system for a time period exceeding the lifespan of the TACs under conditions suitable for expanding the limbal epithelial progenitor cells.

In a typical method, the mixture of limbal progenitor cells and TACs is obtained from donor (e.g., human) limbal corneal epithelial sheets isolated by digestion with an appropriate protease (e.g., Dispase II) in an appropriate medium (e.g., KSFM at 37° C. for 2 h) from the corneoscleral ring (see Espana et al., Invest. Ophthalmol. Vis. Sci. 44:4275-4281, 2003). This isolated mixture of limbal epithelial progenitor cells and TACs are seeded in an appropriate medium at a density of about 10-500 cells/cm² and cultured for a time period greater than about 3 weeks. In this method, the mixture of cells is seeded in the culture system at a cell density sufficiently low to prevent the TACs from having a negative paracrine effect in the limbal epithelial progenitor cells. The cells are incubated under appropriate conditions for cell expansion (e.g., at 37° C., in a 5% CO₂ humidified incubator, with medium changes as necessary). Expanded cells can be re-seeded into new culture vessels for further expansion. Once expanded to desired numbers, cells can be harvested for use. In an alternative embodiment, most TACs can be eliminated by isolating cells from the remaining limbal stroma that is surgically dissected and then digested by 2 mg/ml collagenase A solution in serum-free KSFM medium at 37° C. for 16 h (see Kawakita et al., Am J Pathol. 167:381-393, 2005) and cultured on plastic dishes in KSFM at a seeding density of approximately 10,000 cells/cm².

### Treatment of Diseases

Animal cells expanded ex vivo according to systems and methods described herein can be transplanted into an animal subject suffering from any of a number of disease states in which stem cells are dysfunctional or lacking. For example, an animal subject suffering from HIV or cancer in which stem cells are dysfunctional can receive a transplantation of stem cells expanded ex vivo to restore the function of the dysfunctional stem cells. As another example, cells expanded according to the methods described above can be used to replace cells lost due to HIV infection or cancer or due to the side effects of treatment for those conditions (e.g., cell death caused by anti-viral or anti-neoplastic drugs or ionizing radiation).

### EXAMPLES

The examples set forth below describe controlling the differentiation of primary cells explanted from an animal or human subject such as corneal keratocytes and limbal epithelial progenitor cells from a variety of mammalian species including human beings. Because TGF-β signaling is conserved among a large variety of different cell types, the results set forth below and methods described herein can be adapted for other cell types with minor modifications.

### Example 1

### Preservation and Expansion of Primate Keratocyte Phenotype by Downregulating TGF-β Signaling in a Low Calcium Serum-Free Medium

### Methods

Three rhesus monkeys (Macaca Mulatta), 4 years old, and rabbit and mouse cornea were obtained from an approved tissue-sharing program after euthanasia. An entire anterior corneoscleral segment was removed from the globe by cutting near the limbus with Wescott's scissors. A central cornea was obtained with an 8.0 mm Barron's trephine and immediately transferred to KSFM medium (cat# 17005-042, GIBCO Invitrogen corporation, Carlsbad, Calif.). After removing Descemet's membrane and the corneal endothelium, the corneal epithelium was removed by dispase digestion for 16 h at 4° C. and the remaining corneal stroma was incubated at 37° C. for 16 h in 2.5 ml of DMEM containing 1 mg/ml collagenase A, 20 mM HEPES, 50 μg/ml gentamicin and 1.25 μg/ml amphotericin in a plastic dish. Afterwards, corneal stromal cells were resuspended in 1 ml of KSFM, centrifuged to remove residual matrices, resuspended in KSFM, and seeded on plastic dishes in KSFM or DMEM containing insulin, transferrin, and selenium supplement (DMEM/ITSG) (cat# 41400-045, GIBCO, Carlsbad, Calif.) or 10% FBS (DMEM/10% FBS).

When the primary culture on plastic reached 80% confluence, cells were rendered into single cells by incubation in BSS containing 0.25% trypsin/1 mM EDTA at 37° C. for 1 to 5 minutes, and the enzymatic reaction was stopped by adding soybean-trypsin inhibitor. After centrifuging at 800×g for 5 minutes, cells were resuspended in KSFM, subdivided into 3 equal parts and seeded on plastic dishes. They were cultured in KSFM continuously until use. Keratocytes were similarly isolated from mouse, rabbit and human corneas and cultured in KSFM for comparison.

To verify cell proliferation in KSFM, primary cells in DMEM/ITS, DMEM/10% FBS and KSFM were subcultured at a density of 3,000 cells per 96-well plastic dish, and submitted at day 3 and day 7 to MTT assay (Promega Corporation, Madison, Wisc.) according to the manufacturer's instructions. Using the culture medium alone as the negative control, this assay was validated by establishing a linear correlation between 2,500 and 10,000 passage 2 murine corneal fibroblasts. Cells at day 7 were also immunostained using an anti-Ki67 antibody (1:100). The number of Ki67 positive nuclei was randomly measured in 10 fields under high magnification (400×) for each culture, and the ratio of positive cells/total cells at each field was calculated. Experiments were performed in triplicate. Statistical analysis was performed by using Student's t-test. P<0.05 was considered statistically significant.

Freshly isolated cells expanded in KSFM were subcultured on plastic and upon 60-80% confluence cells at passage 1 (P1) were transferred to a dish in KSFM and continuously cultured and subcultured. As a comparison, cells were also subcultured on a dish in which the [Ca²⁺] was increased to 1.8 mM in KSFM by adding CaCl₂ with or without 10% FBS, or changed to DMEM/10% FBS to examine the ill effect of increasing Ca concentrations and/or addition of FBS. To determine the cell phenotype, cells were transfected for 24 h with self engineered aden-track-Kerapr3.2-intron-ECFP/BpA adenovirus at an MOI of 200 (see Kawakita et al., J Biol. Chem. 280:27085-27092, 2005). To examine the TGF-β signaling, the medium of P1 cells was replaced with fresh KSFM, DMEM/ITS, or DMEM/10% FBS 5 h before being transfected with replication-defective adenoviruses containing TGF-β1 or TGF-βRII promoters, each linked with luciferase (100 MOI) and containing CMV-β-galactosidae (30 MOI) for 48 hours. The promoter activity was measured by the Luciferase Assay Systems (Promega, Madison, Wisc.) and normalized with the β-galactosidase activity. In the same manner, TGF-β1 and -β RII promoter activity of P1 cells was measured in cells cultured in KSFM, in which the [Ca²⁺] was increased to 1.8 mM (identical to DMEM) with or without 10% FBS. β-galactosidase activity was measured and relative transfection was normalized.

### Results

The cellular morphology of rhesus monkey keratocytes in vivo was studied by phase contrast microscopy and LIVE/DEAD assay®, of which the latter demarcated the entire cytoplasm. The monkey keratocytes showed a compact cell body with long dendritic cytoplasmic processes connecting with neighboring cells. These processes formed extensive intercellular contacts in a three dimensional pattern. In addition, CD34 was clearly expressed in the cytoplasm of these cells using both immunohistochemistry and immunofluorescence staining. Western blot analysis showed that an affinity-purified polyclonal antibody against human keratocan also cross-reacted with monkey keratocan. This cross-reactivity was attributed to the fact that there is 92.5 to 95% of homology between human and rhesus monkey keratocan genes. Western blot analysis showed a smear of high MW region in undigested samples consistent with the nature of proteoglycans, and a major band at 56 kDa in endo-β-galactosidase-digested monkey corneal stromal extracts. Using this antibody, keratocan was found to be expressed by keratocytes and the extracellular matrix in the entire monkey corneal stroma, but not by the corneal epithelium nor the corneal endothelium. These results showed that rhesus monkey keratocytes had a dendritic morphology and extensive cell-cell contacts, and expressed both CD34 and keratocan.

The monkey corneal stroma was subjected to collagenase digestion. The resultant cell suspension yielded approximately 1.5×10⁵ cells per cornea. Within 24 hours after seeding on plastic, cells attached well in DMEM/ITS, DMEM/10% FBS, or KSFM, but exhibited a distinctly different morphology. Cells cultured in DMEM/ITS for 7 days did not grow and showed a mixture of flattened and dendritic cells, while cells cultured in DMEM/10% FBS for 7 days reached confluence and showed a flattened fibroblastic morphology. In contrast, cells cultured in KSFM for 7 days had a higher cell density than DMEM/ITS and maintained a dendritic morphology.

To determine whether the dendritic morphology of keratocytes could be similarly maintained in KSFM in other species, primary cells were isolated from human, rabbit and mouse corneal stroma in the same manner and were cultured in KSFM on plastic for 48 hours. All cells showed prominent dendritic processes and extensive intercellular contacts similar to what was shown in monkey keratocytes. Both rabbit and human cells had a triangular cell body and longer dendrites; mouse cells had a rounder cell body and had thinner dendrites. These data showed that the dendritic morphology could be similarly maintained in KSFM for primary cultures of monkey, human, rabbit and mouse keratocytes.

To verify that cells were indeed proliferating in KSFM, an MTT assay was performed at day 3 and day 7 and immunostaining of Ki67 in primary cells at day 7. The number of cells measured by MTT was not significantly changed when cells were cultured in DMEM/ITS, but significantly increased when cultured in DMEM/10% FBS from day 3 to day 7 (p<0.01). The cell number in KSFM estimated by MTT was between that of DMEM/ITS and DMEM/10% FBS. (p<0.05, between day 3 and day 7). When assayed by the proportion of positive Ki67 nuclei, cellular proliferation in KSFM was also between that in DMEM/10% FBS and that in DMEM/ITS (p<0.05, both between KSFM and DMEM/10% FBS or DMEM/ITS). Cells maintained in DMEM/ITS could not be subcultured to P1. They immediately adopted a flattened morphology when subcultured in DMEM/10% FBS at P1. In contrast, cells subcultured in KSFM continued to maintain a dendritic morphology at P8 and P15. These results indicated that cells continued to maintain a dendritic morphology on plastic so long as cultured in KSFM. They reached the number of approximately 2.0×10⁵ in a 60 mm dish at each passage.

Because in vivo monkey keratocytes expressed keratocan and CD34, whether keratocan and CD34 proteins were continuously expressed by dendritic cells that were maintained at late passages in KSFM was examined, and whether such expression could be altered if the medium was switched to DMEM/ITS or DMEM/10% FBS was examined. When P14 cells cultured in KSFM were subcultured in DMEM/ITS or DMEM/10% FBS for 14 days, the dendritic morphology was changed to a flattened (fibroblastic) shape. In contrast, cells continuously subcultured in KSFM still maintained a dendritic morphology. Immunostaining revealed that expression of keratocan was markedly attenuated when subcultured in DMEM/ITS or DMEM/10% FBS, but continued in KSFM. Similarly, expression of CD34 was markedly downregulated when subcultured in DMEM/ITS or DMEM/10% FBS, but continued in KSFM. Because ALDH was a marker of human keratocytes, it was found that ALDH was expressed in primary cells cultured in DMEM/ITS, but lost in cells cultured in DMEM/10% FBS, but maintained in cells cultured in KSFM. Expression of ALDH was similarly downregulated when P14 keratocytes were subcultured in either DMEM/ITS or DMEM/10% FBS. These results indicated that the dendritic morphology of monkey keratocytes correlated well with expression of keratocan, CD34 and ALDH and such a phenotype could be maintained in KSFM, but lost when the medium was switched to either DMEM/10% FBS or DMEM/ITS.

KSFM is culture medium supplemented by growth factors including EGF and bFGF, and differs from DMEM-base medium in many aspects; the major features of KSFM are a low [Ca²⁺] and the lack of FBS. Whether high [Ca²⁺] or addition of 10% FBS or a combination of both might modulate the keratocyte phenotype determined by expression of keratocan was thus examined. To do so, the promoter activities following transient transfection of Aden-track-Kerapr3.2-intron-ECFP/BpA adenovirus containing CMV promoter-driven EGFP and keratocan promoter-driven ECFP in P1 cells was measured. In a given cell, expression of EGFP reflects the background transfection while expression of ECFP reflects the keratocan promoter activity. The protein expression of keratocan and CD34 was also monitored by immunostaining.

Compared to the dendritic morphology of keratocytes cultured in KSFM, most cells remained dendritic, but some cells became flattened in KSFM when [Ca²⁺] was increased to 1.8 mM. In contrast, the majority of cells lost the dendritic morphology and became flattened when 10% FBS was added in KSFM with low [Ca²⁺] or with high [Ca²⁺]. The percentage of ECFP-expressing cells to EGFP-expressing cells of the control cultured in KSFM alone was 70.3±9.2% (mean±s.d.). Such a percentage decreased to 62.0±9.6%, 33.3±5.4% and 29.8±4.5% when cells were cultured in KSFM with 1.8 mM [Ca²⁺], in KSFM with 10% FBS, and KSFM with 1.8 mM [Ca²⁺] and 10% FBS, respectively (p<0.01 for KSFM vs. KSFM+FBS or KSFM+[Ca²⁺]+FBS). There was no significant difference in those percentages between KSFM+[Ca²⁺]+FBS and DMEM/10% FBS nor between KSFM and KSFM+[Ca²⁺]. Immunostaining showed expression of keratocan in cells cultured in KSFM and in KSFM with high [Ca²⁺], but lost in KSFM with 10% FBS and in KSFM with high [Ca²⁺] and 10% FBS. Expression of CD34 was observed in cells cultured in KSFM and KSFM with high [Ca²⁺], but lost in KSFM with 10% FBS and in KSFM with high [Ca²⁺] and 10% FBS. These results indicated that the keratocyte phenotype was not significantly affected in KSFM by increasing [Ca²⁺], but was lost by addition of FBS. The latter detrimental effect was synergistic with increasing [Ca²⁺].

Whether TGF-β signaling was also similarly modulated by increasing [Ca²⁺] or addition of 10% FBS, or a combination of both in KSFM was examined by measuring the promoter activity of TGF-β1 and -β RII after transient adenoviral transfection. As compared to the control, i.e., cells cultured in DMEM/FBS10% and adjusted by background transfection with CMV-β Gal, the promoter activity of TGF-β1 and -β RII was both significantly decreased in cells cultured in KSFM (p<0.05). There was no significant difference in the promoter activity between KSFM and DMEM/ITS. Compared to the control cultured in KSFM alone, increased [Ca²⁺] or addition of 10% FBS did not change the promoter activity for both TGF-β1 and -β RII (p>0.05). In contrast, a combination of increased [Ca²⁺] and addition of 10% FBS significantly upregulated the promoter activity for TGF-β1 and -β RII (p<0.05 and p<0.01, respectively). These results further supported the notion that the loss of keratocyte phenotype with respect to the dendritic morphology and expression of keratocan and CD34 as a result of increased [Ca²⁺] and addition of 10% FBS was correlated with upregulation of the transcriptional activity of TGF-β1 and -β RII genes.

To determine whether the aforementioned phenotype changes and suppression of transcription of TGF-β1 and TGF-β RII genes were correlated with change of Smad-mediated signaling, immunostaining of Smad2 and Smad4 was performed. The majority of cells cultured in DMEM/ITS or KSFM showed cytoplasmic localization of Smad2 and Smad4, while the majority of cells cultured in DMEM/10% FBS or KSFM with increased [Ca²⁺] and addition of 10% FBS showed nuclear localization of Smad2 and Smad4. The percentage of cells exhibiting nuclear accumulation of Smad2, an index suggestive of phosphorylation of Smad2, was 38±7.6% (mean±s.d.) in DMEM/ITS and 88.7±4.0% in DMEM/10% FBS, of which both were significantly higher than 19.3±5.1% in KSFM (p<0.01). Even when 4 ng/ml TGF-β1 was added in KSFM for 48 hours, the percentage of nuclear accumulation of Smad2 in cells increased to 34.7±4.9%, which was still not higher than that of DMEM/ITS (p>0.05). Similarly, the percentage of nuclear accumulation of Smad4 was 27.7±1.5%, 90.7±2.1%, and 12.0±3.0% in DMEM/ITS, DMEM/10% FBS, and KSFM, respectively. These results indicated that Smad-mediated TGF-β signaling was significantly downregulated in cells cultured in KSFM.

### Example 2

### Keratocan Expression of Murine Keratocytes Is Maintained on AM by Downregulating TGF-β Signaling

Keratocytes display a dendritic morphology and express keratocan. When cultured using conventional methods, however, keratocytes lose their dendritic morphology and cease expression of keratocan. As described below, keratocytes were expanded on AM and examined to determine if they maintained their characteristic phenotype, including the expression of keratocan.

### Materials and Methods

Isolation and Culture of Keratocytes on Plastic or AM—Albino mice eyes were enucleated by forceps, washed profusely in PBS, and incubated in DMEM containing 20 mM HEPES, 15 mg/ml dispase II (Roche, Indianapolis, Ind.) and 100 mM sorbitol at 4° C. for 18 h (see Espana et al., Invest Ophthalmol Vis Sci. 44:4275-4281, 2003; Kawakita et al., Invest Ophthalmol Vis Sci. 45:3507-3512, 2004). The entire corneal epithelium loosened by this treatment was subsequently removed by vigorous shaking. Under a dissecting microscope, the corneal stroma was separated from the sclera at the corneoscleral limbus by pressing down the limbus with a 27 G needle while the eye was held with a forcep. Isolated corneal stromas were incubated overnight at 37° C. in DMEM containing 1.25 mg/ml collagenase A (Roche, Indianapolis, Ind.), 50 μg/ml gentamicin and 20 mM HEPES in a non-coated plastic dish until the tissue became “smeared” onto the dish bottom. Digested corneal stromas in collagenase A were centrifuged at 800×g for 5 min. Keratocytes were resuspended in DMEM containing 20 mM HEPES, ITS (5 μg/ml insulin, 5 μg/ml transferrin and 5 ng/ml sodium selenite), 50 μg/ml gentamicin and 1.25 μg/ml amphotericin B with or without 10% FBS. This keratocyte-containing cell suspension was then seeded on plastic dishes or on the stromal side of the AM (Bio-Tissue, Miami, Fla.) fastened to a culture insert as previously described (see Meller et al., Br J Ophthalmol 86, 463-471, 2002).

The suspension of keratocytes prepared from 3-4 murine corneal buttons was seeded on a 35 mm plastic dish or on the stromal surface of one 32 mm AM insert (see Espana et al., Invest Ophthalmol Vis Sci 44, 5136-5141, 2003). Cells were cultured in DMEM supplemented with 10% FBS (DMEM/10% FBS), and the medium was changed every 2-3 days. When cells reached 80-90% confluence, they were dissociated into single cells by incubation in 0.05% trypsin and 0.53 mM EDTA in HBSS at 37° C. for 5 min in plastic dishes or for 20 min in AM inserts, followed by vigorous pipetting. After centrifuging at 800×g for 5 min, cells were resuspended in DMEM/10% FBS and seeded on a plastic dish or AM stroma. They were cultured in DMEM containing 10% FBS, 20 mM HEPES, 50 μg/ml gentamicin and 1.25 μg/ml amphotericin B.

TGF-β1 Challenge and Neutralizing Antibody—To assess whether TGF-β1 affected the cell phenotype, triggered Smad 2 and Smad 4 nuclear translocation, and differentiated keratocytes into myofibroblasts, 10 ng/ml human recombinant TGF-β1 (Sigma, St Louis, Mo.) was added to serum-free DMEM/ITS cells for 3 h or 5 days when cells expanded on AM were passed to 24 well plastic dishes and AM inserts, respectively. In addition, primary keratocytes were seeded and cultured on AM or plastic for 3 days in DMEM/ITS or DMEM/10% FBS for 24 h, of which the latter was treated with or without 10 μg of a monoclonal antibody neutralizing TGF-β1, -β2, and -β3 (R&D Systems, Minneapolis, Minn.) per ml of DMEM medium for 48 h before adenoviral transfection.

Assays of Cell Proliferation—To verify that cells indeed proliferated on AM, the passage 2 (P2) cells that were continuously cultured on either AM or plastic were subcultured at a density of 10,000 cells per 24-well plastic dish in DMEM/ITS or DMEM/10% FBS or on AM in DMEM/10% FBS. Cells were terminated at day 3 and day 7 for MTT assay (Roche, Nutley, N.J.) according to the manufacturer's instruction. This assay measured by absorbance at 550 nm yielded a linear correlation for cell numbers above 2,500 cells using P2 murine corneal fibroblasts. Cells at day 7 were also immunostained using an anti-Ki67 antibody. The number of Ki67 positive nuclei was randomly measured in 10 fields under high magnification (400×) for each culture. Experiments were performed in triplicate.

Transient Transfection and TGF-β Promoter Assays—Freshly isolated cells expanded on AM were subcultured on plastic and AM inserts. Upon reaching 60-80% confluence, cells in each 24 well plate or AM insert were co-transfected with 1.0 μg/ml plasmid DNA containing TGF-β2 or TGF-β RII promoter-luciferase and 1.0 μg/ml pCMV/Sport/Bgal (Invitrogen, Carlsbad, Calif.) using GeneJammer® (Stratagene, LaJolla, Calif.) according to manufacturer's protocol.

### Results

In Vivo Morphology and Keratocan Expression of Murine Keratocytes—In CD-1 albino murine globes, there is a visible boundary that demarcates the limbus between the cornea and the sclera. Such a demarcation facilitated the surgical isolation of the corneal stroma. Following the removal of an intact sheet of the ocular surface epithelium from the globe by Dispase II, the corneal stroma was dissected from the adjacent sclera. Live/Dead Assay® revealed that an overwhelming majority of keratocytes were viable and exhibited a 3-dimensional dendritic morphology in the corneal stroma. Some dead cells were found in the cut edge of the excised stroma. Keratocytes in the stroma expressed keratocan as evidenced by positive staining with an affinity-purified antibody against mouse keratocan peptide. In contrast, corneal epithelial cells or endothelial cells were not stained. These results indicated that in vivo murine keratocytes also exhibited a characteristic dendritic morphology and specifically expressed keratocan.

In Vitro Morphology and Proliferation of Keratocytes Cultured on AM—Keratocytes were then isolated from the corneal stroma by collagenase digestion. An average of 5,000 cells was obtained per mouse cornea. Cells at a density of 3,000 per cm² were seeded on either plastic or AM in DMEM/10% FBS. Within 12 h after seeding, cells attached on either substrate, and exhibited a distinctly different morphology. Cells on plastic dishes were evenly distributed on the flat surface and adopted a spindle-shaped morphology with a broad stellate cytoplasm. They became confluent in 4 to 5 days. In contrast, cells on AM were dendritic or satellite in shape and had a triangular cell body and a scanty cytoplasm which formed extensive intercellular networks, and projected their dendritic processes in a 3-dimensional manner. They became confluent in 10 days.

Upon reaching 80-90% confluence, cells expanded on AM or plastic were trypsinized and continuously passaged onto the same type of substrate as used in the primary culture (P0). Cells subcultured on plastic at P1 became more flattened. In contrast, cells expanded on AM subcultures still maintained a dendritic morphology with pronounced intercellular contacts. They continuously preserved such a dendritic morphology until passage 8, when cells became senescent. Using MTT assay, cells of P2 plastic cultures in DMEM/ITS did not show an increase of cell number during the one week of culturing, while cells on plastic in DMEM/10% FBS rapidly expanded in number. Cells cultured on AM in DMEM/10% FBS were intermediate between that of the above two conditions (p<0.05 cf. DMEM/ITS and p<0.01 cf. DMEM/10% FBS). At day 7, the number of Ki67 positive nuclei in cells cultured on AM in DMEM/10% FBS was significantly more than that of cells on plastic in DMEM/ITS, but less than cells cultured on plastic in DMEM/10% FBS (both p<0.01). Collectively, these results confirmed that cells cultured on AM continued to proliferate and maintained a dendritic morphology in a FBS-containing medium.

Phenotypic Characterization of Cells Expanded on AM—To confirm that dendritic cells expanded on AM were indeed keratocytes and not myofibroblasts, immunostaining was performed for the expression of keratocan and α-SMA, respectively. For primary cultures (P0), a majority of dendritic cells cultured on plastic in DMEM/ITS for 5 days expressed keratocan but not α-SMA. In contrast, cells cultured on plastic in DMEM/10% FBS were not dendritic and did not express keratocan; instead some cells expressed α-SMA. However, dendritic cells expanded on AM in DMEM/10% FBS maintained keratocan expression, and did not express α-SMA. In DMEM/10% FBS, CD34 was not expressed by cells cultured on plastic, but expressed by cells cultured on AM. In contrast, fibronectin was expressed extracellularly and intracellularly by cells cultured on plastic, but not expressed by cells cultured on AM. These results collectively indicated that the keratocyte phenotype was maintained by AM.

To confirm transcript expression of keratocan, total RNAs were extracted from cells on plastic and AM, and subjected to RT-PCR. The results showed that keratocan transcript (of the size of 1065 bp) was expressed by cells cultured on plastic at passage 0, but lost at passage 1 and thereafter. In contrast, the keratocan transcript was continuously expressed in an abundant amount from passage 0 to passage 3 and up to passage 8 when cultured on AM.

To verify the keratocan protein expression, insoluble matrix proteins were extracted by 4 M guanidine HCl and subjected to Western blot analysis using an antibody against the core protein of keratocan. The sample from the normal murine corneal stroma, which was used as the positive control, showed a dense smearing in the high molecular weight region. Nevertheless, the same sample after digestion with endo-β-galactosidase showed a positive protein band of ˜50 kDa. The undigested sample of AM cultures at passages 6 and 8 showed a similar faint smearing in the same high molecular weight region. Both samples after digestion with endo-β-galactosidase showed a strong positive protein band of 50 kDa. A similar 50 kDa band was obtained from P2 cultures on AM, but not from P2 cultures on plastic using digestion by keratanase II. In contrast, there was no smearing in the undigested sample, nor was the protein band detected after digestion in plastic cultures at passage 1 and thereafter. The negative control of pure AM extract alone without any cultured cells did not contain any keratocan without or with endo-β-galactosidase digestion. Because keratocan expression was strongly observed in the extracellular matrix of in vivo murine corneas, conditioned media from P2 murine keratocyte cultures was also examined for keratocan expression. The results showed that the digested samples of the conditioned medium from AM culture, but not plastic cultures, showed a 50 kD band. Collectively, these data indicated that in DMEM/10% FBS cells expanded on AM, but not plastic, expressed keratocan in the matrix and the conditioned medium.

Transient and Sustained Suppression of Smad-dependent TGF-β Signaling in Keratocytes Cultured on AM—10 ng/ml TGF-β1 was added to both plastic and AM cultures containing DMEM/ITS and the Smad-mediated TGF-β signaling was examined by immunolocalization of Smad 2 and Smad 4. Cells on AM or plastic responded differently to exogenous addition of TGF-β1. Nuclear localization of Smad 4 was found in 16% of cells on plastic without TGF-β1, but increased to 67% and 85% of cells cultured on plastic after 3 h and 5 days of TGF-β1 challenge, respectively. A similar trend was noted for nuclear localization of Smad 2. In contrast, no cell cultured on AM showed nuclear localization of Smad 2 and Smad 4 at 3 h and 5 days of TGF-β1 challenge. These results indicated that TGF-β signaling mediated by Smad was continuously suppressed in cells cultured on AM even in the presence of 10% FBS.

To confirm that TGF-β was indeed responsible for Smad signaling in DMEM/10% FBS, a neutralizing antibody to three TGF-β isoforms was added to the plastic cultures. Nuclear translocation of Smad 4 was prevented. To demonstrate whether nuclear localization of Smad 4 also correlated with downstream of TGF-β signaling, α-SMA expression was quantified in parallel. Thirty-nine percent of cells cultured on plastic differentiated into α-SMA-expressing myofibroblasts, but no cell on AM expressed α-SMA even after 5 days of continuous stimulation with TGF-β1. Taken together, these results demonstrated that Smad-mediated TGF-β signaling was inhibited in cells cultured on AM and suppression of Smad-mediated TGF-β signaling correlated with prevention of cells from differentiating into myofibroblasts.

Inhibition of TGF-β2 and TGF-βRII Transcriptional Activity in Keratocytes Cultured on AM—To determine whether the aforementioned downregulation of TGF-β signaling was mediated by suppressing TGF-β genes at the transcriptional level, TGF-β2 and TGFβ-RII promoter activities were evaluated by transient transfection. As compared to cells cultured on plastic and adjusted by background transfection with CMV-β Gal, the promoter activity of TGF-β2 and TGF-β RII was decreased 4.1-fold and 2.6-fold, respectively, in cells cultured on AM (both p<0.001). These data suggest that down-regulation of TGF-β signaling was indeed mediated by suppressing TGF-β2 and TGF-β-RII genes at the transcriptional level for cells expanded on AM.

Suppression of TGF-β Signaling Maintained Keratocan Expression—To demonstrate a direct link between downregulation of TGF-β signaling and keratocan expression, 50 multiplicity of infectivity (M.O.I.) of Aden-track-Kerapr3.2-intron-ECFP/BpA was added to cells cultured on plastic in either DMEM/ITS or DMEM/10% FBS, of which the latter was further treated with or without an antibody to neutralize all three TGF-β isoforms. Transfection efficiency was revealed by EGFP (green fluorescence) driven by CMV in the same construct, while expression of keratocan promoter was revealed by ECFP (blue fluorescence) in the same cell. Cells retained the dendritic morphology after transfection in the positive control cultured on plastic in DMEM/ITS or on AM in DMEM/10% FBS. Cells also maintained a flattened bipolar morphology in the negative control cultured on plastic in DMEM/10% FBS. These results indicated that transfection itself did not alter their respective characteristic cell morphology. Interestingly, the fibroblastic morphology did not revert to a dendritic morphology after the addition of TGF-β neutralizing antibody for 2 days. The overall transfection efficiency was more than 80% in these experiments. Under such a high transfection rate, keratocan promoter-driven ECFP expression was observed in 30-40% of cells cultured on plastic in DMEM/ITS, 15-20% of cells cultured on AM in DMEM/10% FBS, but less than 2% of cells cultured on plastic in DMEM/10% FBS. These results corroborated with the aforementioned pattern of keratocan transcript and protein expression in these three cultures. ECFP expression was restored to 10-15% of cells cultured on plastic in DMEM/10% FBS when TGF-β neutralizing antibody was added. Collectively, these results indicated that TGF-β in 10% FBS was indeed responsible for the suppression of keratocan expression for cells cultured on plastic, and that keratocan expression by cells cultured on AM in 10% FBS was correlated with suppression of Smad-mediated TGF-β signaling.

### Example 3

### Clonal Initiation and Expansion of Murine Limbal Progenitor Cells In a Fibroblast-Free, Matrix-Free, and Serum-Free Niche

A flat mount preparation of freshly isolated intact human limbal epithelial sheet showed that p63-positive (p63 being an epithelium-specific transcription factor) basal cells are grouped in clusters, indicating that progenitor cells are intermixed with TACs in the limbal basal epithelium. Because TACs are known to have a negative paracrine influence on limbal epithelial progenitor cell renewal, it was hypothesized that elimination of TAC's paracrine influence by seeding at a low density and prolonging the culturing time beyond TAC's life span (about 3 weeks) would improve clonal initiation and expansion of limbal epithelial progenitor cells. Single cells dissociated from isolated mouse corneal/limbal sheets by trypsin/EDTA were seeded at a density of 40 cells/cm² in a defined keratinocyte serum-free medium (KSFM) (Gibco-BRL, Carlsbad, Calif.) containing 0.07 mM (low) Ca²⁺ but supplemented with insulin, bFGF, EGF and cholera toxin.

When cells were seeded at a low density of 40 cells/cm², no cell growth was noted for the first 3 wks (i.e., within TAC life-span). On day 25 (>3 wks) there emerged an average of 2 to 3 large clones (stained by crystal violet) per 60 mm dish, a frequency similar to that seeded on mitomycin C-arrested 3T3 fibroblast feeder layers (which had more smaller clones). The large clone had a smooth perimeter and consisted of small epithelial cells, resembling “holoclone”, which has been used to denote epidermal stem cells and limbal progenitor cells. Expression of K12 keratin was negative in KSFM but positive in 3T3 fibroblast cultures, suggesting that KSFM is more ideal for maintaining stemness (stem cell characteristics) because cellular differentiation is less promoted than in 3T3 fibroblast cultures.

Elimination of TAC's paracrine influence by seeding at a low density and by prolonging the culturing time beyond TAC's life span (>3 wks) improved eliciting clonal initiation and expansion of limbal progenitor cells. Using this technique, clones were generated that could continually be expanded for more than 25 passages for a period of nearly two years (each passage spanning for one month). Two types of clonal growth (one fast and the other aborted) could be generated from a single cell derived from these holoclones by limiting dilutions in a 96 well culture plate. Several such single-cell generated clones were expanded, and each proved to be non-transformed. These single cell-generated clones could be cryopreserved and then recovered more than once.

During the early stage of the clonal growth (i.e., before Day 14) nearly all cells were uniformly small and round (<10 μm), negative for K12 keratin expression, and positive for p63 and K14 keratin expression. After Day 14, some cells (especially in the periphery) became enlarged, negative for p63 expression, and positive for K12 keratin and α-smooth muscle actin expression. Elevating extracellular calcium concentration ([Ca²⁺]) to 0.9 mM and/or adding 5% FBS caused the cells to become enlarged and squamous, to express K12 keratin, and lose expression of p63. These treatments also increased the level of TGF-β in the conditioned medium when TAC differentiation appeared.

The foregoing technique was also successfully used to isolate similar small cells from human limbal epithelial sheets, umbilical cord epithelium, and human amniotic epithelium. For human limbal epithelial progenitor cells, the same results were obtained using the above technique except that the cell seeding density could be as high as 2,500 cells/cm². An alternative method to eliminate most of the TACs in the example of expanding human limbal epithelial progenitor cells is to surgically dissect the remaining limbal stroma from the sclera after dispase digestion as stated above, and then to digest it by 2 mg/ml collagenase A solution in KSFM medium at 37° C. for 16 h (Kawakita et al., Am J Pathol. 167:381-393, 2005). Cells thus isolated were then cultured on plastic dishes in KSFM at a seeding density of 10,000 cells/cm². For human amniotic epithelial progenitor cells, the same results were obtained using the above technique with the exception that Ca²⁺ concentrations could be elevated as high as 1.0 mM. For all of the above epithelial cell cultures, adding 5% FBS and increasing the Ca²⁺ concentration to 1.8 mM caused rapid differentiation of these cells. By isolating limbal epithelial progenitor cells from deep within the stroma, TACs can be avoided, as TACs are not located deep within the stroma.

To show that the ability of KSFM, a low-calcium, serum-free medium, to promote limbal epithelial progenitor cell isolation and expansion resides in its ability to suppress TGF-β signaling, adenoviral vectors containing the promoters of TGF-β1, TGF-β2, TGF-β3, or TGF-βRII and the reporter gene luciferase were constructed and used to monitor the transcriptional activity of these four TGF-β genes in transiently transfected human limbal epithelial progenitor cells. Using this promoter assay, transcription of TGF-β1 and TGF-βRII was found to be markedly downregulated in human limbal epithelial cells when the cells were maintained in KSFM medium when compared to SHEM which contains 5% FBS and high calcium. Addition of 5% FBS to and elevation of [Ca²⁺] to 0.9 mM in KSFM, markedly upregulated TGF-β1 promoter activity to the same level as SHEM in human limbal epithelial progenitor cells and monkey.

These promoter activities were markedly elevated in DMEM with 10% FBS or SHEM (containing DMEM/F12 (1/1) and 5% FBS), both with high Ca⁺² and FBS, but significantly downregulated in KSFM. These promoter activities were significantly downregulated when AME were added in SHEM, suggesting that the capability of AME in suppressing TGF-β signaling can take place even in medium with high Ca⁺² and FBS.

In other experiments, clonally expanded murine epithelial progenitor cells were seeded at 500 cells/cm² (low), 5,000 cells/cm² (intermediate) and 50,000 cells/cm² (high) densities in KSFM. After 6 to 14 days in culture, most cells were small without expressing K12 keratin at the low density, but were large and expressed K12 keratin at the intermediate and high densities. Conditioned media was collected from these three cultures after 3 days of culturing in KSFM and subjected to a Bio-Plex machine (Bio-Rad, Hercules, Calif.) using Beadlyte® TGF-β1, β2, β3 detection system (Upstate, Waltham, Mass.). 4.3±0.5 ng/ml TGF-β1 and 5.1±0.4 ng/ml TGF-β2 (n=3) was detected only in the conditioned media of high density cultures seeded at 5,000 cells/cm² but not in those of low density cultures seeded at 50 or 500 cells/cm². No TGF-β3 was detected. Because the detection limit of this system is down to 0.1 ng/ml, the above data indicated that the levels of TGF-β1 or β2 in the low density cultures should be less than 0.1 ng/ml.

Using the methods described above, umbilical cord epithelial cells were expanded ex vivo while maintaining their stem cell phenotype.

### Example 4

### Amniotic Epithelial Cells Help Maintain HA-containing Stromal Matrix in Intact AM to Support Limbal Epithelial Progenitor Cell Renewal by Downregulating TGF-β Signaling

To determine whether amniotic epithelial cells on intact AM are pushed away or grown over by limbal epithelial cells migrating from the limbal explant, expanded limbal epithelial outgrowth was removed as a sheet from intact or denuded AM, respectively, by the method described in Espana et al., Invest Ophthalmol Vis Sci 44:4275-4281, 2003. Devitalized amniotic epithelial cells were present on intact AM, but absent on denuded AM and remaining stroma. A distinct basement membrane judged by a linear staining to collagen IV, laminin 5 and collagen VII was noted in the outgrowth on intact AM. In contrast, staining to collagen IV and laminin 5 was sporadic and diffuse, while that to collagen VII was negative in the outgrowth on denuded AM. The same result was confirmed in the remaining stroma when the epithelial sheet was removed. The remaining stromal matrix of intact AM was thicker than that of denuded AM (dAM) after expansion. These results indicate that amniotic epithelial cells, although devitalized, still play an active role in modulating epithelial basement membrane assembly.

Because HA is a major component of AM stromal matrix, the presence of HA in intact and denuded AM stromal matrix was examined. Using biotinylated HA binding protein (HABP) to immunolocalize HA, HA was observed to be better preserved in intact AM than denuded AM, suggesting that amniotic epithelial cells may partake in preventing the degradation of HA-containing AM stromal matrix, thus helping to maintain the stromal matrix. The thickness of denuded AM stroma was increased when human corneal fibroblasts were seeded on the stromal side of the AM.

A Western blot analysis showed that HA in AM stromal matrix was covalently linked with inter-α-trypsin inhibitor (ITI). The heavy chains of ITI entered the SDS gel only after HA was digested by hyaluronidase (HAse). The HA-ITI complex was not only present in the insoluble extracts (obtained by 1 M NaCl and 4M guanidine HCl, respectively) but also in soluble AM extract (obtained by homogenization in PBS). The covalent linkage of HA with ITI stabilizes high MW status of HA, preventing HA degradation to small MW in part because ITI is a natural inhibitor of HAse. In a related experiment, AME were digested with or without 50 μg/ml HAse. The presence of ITI in these extracts and their interactions with HA were examined by Western blot. ITI was present in all extracts before HAse digestion, but there were extra bands appearing after digestion. These results suggest that ITI exists in AM in at least two forms: free and HA-bound. Additional western blots revealed that TSG-6, a component important for the formation of HA-ITI complex, was found in soluble fraction and in 4M guanidine HCl-extracted insoluble fraction. Pentraxin (PTX3), a component known to help HA crosslinking, was also found mostly in soluble extracts. Furthermore, thrombospondin, an anti-angiogenic component also known to help HA crosslinking, was found in soluble extracts and 4M guanidine HCl extracts.

### Example 5

### Addition of TGF-β1 Induced Expression of α-Smooth Muscle Actin (α-SMA) In Murine Epithelial Progenitor Cells Via Activation of Both Smad and β-catenin/LEF-1 Signaling Pathways

In clonal cultures of murine limbal epithelial progenitor cells, expression of α-SMA, a marker for myofibroblasts, was not detected when clonally expanded murine limbal epithelial cells were seeded at low density (500 cells/cm²), but increasingly detected when cells were seeded at the intermediate (5,000 cells/cm²) and high seeding densities (50,000 cells/cm²). Cells expressing α-SMA were larger and squamous and could also express p63 in the nucleus. Addition of 5 ng/ml TGF-β1 to KSFM upregulated α-SMA expression in cultures seeded at the low density. In contrast, addition of 10 μg/ml of a neutralizing antibody against three TGF-β isoforms significantly reduced α-SMA expression in cultures seeded at the high density. α-SMA was not expressed in the center, but was expressed in the periphery of the single cell-derived clone after being expanded more than 3 weeks. Under these conditions, expression of S100A shifted from cytoplasm to the nucleus, signaling of Smad 4 moved from cytoplasm to the nucleus, expression of E-cadherin moved from the intercellular junction to the cytoplasm and the perinucleus, the signaling of β-catenin moved from the intercellular junctions to the nucleus), and the signaling of LEF-1 also moved from the cytoplasm to the nucleus. These results suggest that the irreversible epithelial-mesenchymal transition can take place in limbal epithelial progenitor cells by expression of α-SMA and S100A4 and is correlated with activation of Smad-mediated signaling and β-catenin/LEF-1 mediated signaling.

### Example 6

### A TGF-β Promoter Assay That Demonstrates the Suppressing Effect of TGF-β Signaling By AME In Both Human Limbal Epithelial Progenitor Cells and Human Corneal Fibroblasts

Human corneal fibroblasts were transiently transfected with the aforementioned adenoviral promoter constructs. In these cells, TGF-β1 promoter activity was significantly suppressed by an AME prepared according to the method described in U.S. provisional patent application filed Mar. 2, 2005 and entitled “Suppression Of TGF-B Activity By Amniotic Membrane Extracts, Compositions Thereof, And Methods For The Prevention And Suppression Of Scarring And Inflammation” in a dose-dependent manner (from 0.04 to 125 μg/ml) in human corneal fibroblasts. In human limbal epithelial cells, TGF-β1 promoter activity was significantly suppressed by 25 μg/ml AME in both KSFM and SHEM media. Furthermore, the suppressive activity of AME (which contained ˜0.8 μg/ml HA) was more potent than 125 mg/ml high MW pure HA alone. The suppressive effect of both AME and HA alone was lost after pretreatment with HAse. No suppressive effect was noted in the control when HAse alone was added together with BSA. A similar result was obtained for TGF-βRII promoter activity.

Single cell expanded murine limbal epithelial cells were seeded at 50 cells/cm² in KSFM and cultured for 6 days. Addition of TGF-β1 at 10 μg/ml and 150 μg/ml dose-dependently suppressed the clonal expansion. In contrast, addition of 10 μg/ml neutralizing antibody against three TGF-β isoforms to the control to suppress endogenous production of TGF-β resulted in expansion of clonal growth with pronounced cell migration. Cells seeded at 20,000 cells/cm² in KSFM became enlarged. However, addition of 125 μg/ml AME promoted expansion of more small cells.

### Example 7

### Signaling Transduction Pathways Required for Ex Vivo Expansion of Human Limbal Explants on Intact AM

The results described below show that ex vivo expansion of human limbal epithelial progenitor cells on intact AM is mediated by the survival signaling pathway mediated by PI3K-Akt-FKHRL1 and the mitogenic MAPK pathway mediated by p44/42, but not by p38 and JNK.

### Methods

Human AM was provided by Bio-Tissue (Miami, Fla.) and stored at −80° C. before use. AM was devitalized by freezing and thawing and washed three times with HBSS before being fastened onto a 30 mm culture insert, Millicel-CM (which generated an insert with 23 mm diameter covered by AM), and placed in a 6 well plate (Meller, D. and Tseng SCG, Invest Ophthalmol Vis Sci., 40:878-86, 1999).

After removal of excessive sclera, iris, corneal endothelium, conjunctiva, and Tenon's capsule, the limbal ring was separated by a 7.5 mm trephine from donor human corneas. Each limbal ring was rinsed 3 times with SHEM media. The limbal ring was then exposed for 10 min to 1.2 units/ml Dispase II in Mg²⁺- and Ca²⁺-free HBSS at 37° C. under 95% humidity and 5% CO₂. Following three rinses with SHEM medium, each limbal ring was subdivided into two halves and each half further subdivided into 6 pieces of 1×1.5×2.5 mm explants. To eliminate variations of age, sex, and race, explants from the corresponding position of the same donor cornea were selected for the control and the experimental group, respectively. An explant was placed on the center of intact AM or plastic with the epithelial side facing up and cultured in SHEM medium. The experimental group was added with the inhibitor of desired concentration, while the control group was added with the same concentration of DMSO as the vehicle which was used to dissolve each inhibitor. The culture was maintained at 37° C. under 95% humidity and 5% CO₂, the medium was changed every other day, and their outgrowth was monitored daily for 17 days using an inverted phase microscope (Nikon, Japan). The outgrowth area was digitized every other day by Adobe Photoshop 5.5 and analyzed by NIH ImageJ 1.30v (NIH, Bethesda, Md.).

All experiments were performed at least in triplicate. Summary data were reported as means±S.D., compiled and analyzed by MicroSoft Excel™ (MicroSoft, Redmont, Wash.). The mean and standard deviation were calculated for each group using the appropriate version of Student's unpaired t-test. Test results were reported as two-tailed p values, where p<0.05 was considered statistically significant.

### Results

To ensure that the control without treatment had a consistent growth rate and pattern, a total of 33 limbal explants from 11 donors ranging 37 to 61 years old were examined. Under microscopic observation, it was noted that epithelial cells started to migrate from the limbal edge to AM in 28 of 33 explants (85%) at day 3-4, while from the corneal or scleral edge in the rest. At day 5, cell outgrowth could be discerned by the naked eye. The surface area was scanned and digitized every other day until day 17 when the outgrowth reached ˜80% confluence, i.e., ˜340 mm² of 415 mm² of the AM insert. The culture was terminated before reaching confluence to avoid possible underestimation caused by cell contact inhibition. The outgrowth rate of the control showed a consistent pattern as a group. The outgrowth rate was gradually increased from day 5 to day 9, but rapidly increased from day 9 to day 13, and gradually slowed down from day 13 to day 17.

The PI3K-Akt pathway controls cell survival, and inhibition of this pathway frequently leads to apopotosis. LY294002 is a specific inhibitor of PI3K, and one of the downstream target of PI3K is to phosphorylate and activate Akt kinase. The epithelial outgrowth was not significantly inhibited by 5 and 10 μM of LY294002 (p=0.85 and 0.09, respectively), but was significantly or completely inhibited by 20 and 50 μM of LY294002 (p=0.0008 and 0.0007, respectively). As compared to the control, complete inhibition of epithelial outgrowth was noted at 20 μM and 50 μM of LY294002. Addition of 10 μM of SR13668, a potent phosphor-Akt inhibitor, resulted in 50% reduction of the outgrowth rate from day 5 to day 11, and 60% reduction from then on. Addition of 50 μM of SR13668 completely inhibited the epithelial outgrowth. These results indicated that inhibition of either PI3K or Akt could completely abolish epithelial outgrowth from the limbal explant cultured on AM.

U0126 is a specific inhibitor of MAPK kinase MEK1/2. At 10 μM, it completely inhibited p44/42 MAPK phosphorylation in many cells. When 10 μM of U0126 was added, epithelial outgrowth was noted at day 8, which was significantly more delayed than the control. From day 13 on, the outgrowth from the U0126-treated explants was almost halted. At day 17, the average outgrowth area of the control and U0126-treated group was 334±34.3 mm² and 17.0±3.0 mm², respectively (p=0.0077). SB203580 and JNK inhibitor 1 are specific inhibitors for MAPK p38 kinase and JNK kinase, respectively. Addition of 10 μM of either SB203580 or JNK inhibitor 1 did not change the outgrowth which started at day 5 and reached similar rates when compared to the control. There appeared some promotion of epithelial outgrowth in SB203580-treated explants, but the difference did not reach a statistical difference (p=0.89). Collectively, these results indicated that inhibition of p44/42 MAPK, but not p38 kinases or JNK, of the MAPK family also completely abolished epithelial outgrowth from the limbal explant cultured on AM.

Because addition of LY294002, SR13668, or U0126 led to complete or significant inhibition of ex vivo expansion of limbal epithelial cells, these inhibitors were thus removed after the explants was treated with 50 μM LY294002, 50 μM SR13668, or 10 μM U0126 for 17 days, respectively. It was noted that the inhibition of epithelial outgrowth was reversible because the outgrowth re-initiated in 2 days after the culture medium containing the inhibitor was switched to the fresh medium. However, the outgrowth was resumed at a much slower rate and took a significantly longer time, i.e., 25-30 days to reach ˜80% confluence. The reversible outgrowth from 10 μM U0126 treatment was faster than those that treated with 50 μM LY294002 or 50 μM SR13668 (25 days vs. 30 days to reach ˜80% confluence). These results indicated that such inhibition was reversible and that the progenitor cells in the limbal explant remained viable and could resume proliferation and migration even being treated by these inhibitors for 17 days.

Western blotting analysis was performed to verify that the respective phosphorylation of these kinases was indeed inhibited following the treatment of the aforementioned inhibitors. The results showed that addition of 50 μM of LY294002 or SR13668 abolished phosphorylation of Akt at Thr308 and Ser473. 50 μM SR13668 also abolished while 50 μM LY294002 decreased Thr32 phosphorylation of FKHRL1, a downstream target of Akt. 10 μM U0126 eliminated phosphorylation of Akt at Thr308 and decreased phosphorylation of Akt at Ser473 and FKHRL1 at Thr32. Only 10 μM U0126 abolished phosphorylation of p44/42 MAPK at both Thr202 and Tyr204, while 50 μM LY294002 and 50 μM SR13668 did not change p44/42 MAPK phosphorylation. 10 μM SB203580 and 10 μM JNK inhibitor 1 did increase the phosphorylation of p44/42 MAPK. Interestingly, phosphorylation at Thr180/Tyr182 of p38 MAPK was expressed by cells expanded on plastic, but markedly downregulated in those expanded on intact AM, and abolished with addition of 10 μM SB203580. Likewise, phosphorylation of Thr183/Tyr185 of JUN MAPK was expressed by cells expanded on plastic, but abolished in those expanded on intact AM with or without addition of JNK inhibitor 1. These data collectively further supported that selective activation of Akt and/or p44/42 MAPK without concomitant activation of p38 and JUN MAPKs is uniquely involved in ex vivo expansion of human limbal epithelial progenitor cells on intact AM without 3T3 fibroblast feeder layers.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An ex vivo method of expanding animal cells whose differentiation state is: controllable by modulating TGF-β signaling, the method comprising the steps of:
(a) providing an animal subject comprising cells having a first phenotype;
(b) isolating the cells from the animal subject;
(c) placing the cells in an ex vivo culture system comprising a culture vessel comprising at least one surface and a medium in contact with the at least one surface, the medium being essentially free of intact amniotic membrane and feeder cells; and
(d) culturing the cells in the medium under conditions which downregulate TGF-β signaling in the cells to allow the cells to proliferate while maintaining the first phenotype.

2. The method of claim 1, wherein the cells are differentiated cells.

3. The method of claim 2, wherein the cells are keratocytes.

4. The method of claim 1, wherein the cells are stem cells.

5. The method of claim 4, wherein the stem cells are selected from the group consisting of: limbal epithelial progenitor cells, umbilical cord epithelial cells, and amniotic membrane epithelial cells.

6. The method of claim 5, wherein the stem cells are limbal epithelial progenitor cells.

7. The method of claim 1, wherein the conditions which downregulate TGF-β signaling in the cells comprise culturing the cells in a medium being essentially free of serum and comprising less than about 0.15 mM Ca²⁺.

8. The method of claim 7, wherein the medium is a defined serum-free medium comprising less than about 0.1 mM Ca²⁺.

9. The method of claim 1, wherein the medium comprises serum and a Ca²⁺ concentration greater than about 1.0 mM and the conditions which downregulate TGF-β signaling in the cells comprise contacting the cells with an agent that downregulates TGF-β signaling in the cells.

10. The method of claim 9, wherein the agent downregulates transcription of a TGF-β gene in the cells.

11. The method of claim 9, wherein the agent specifically binds a TGF-β.

12. The method of claim 11, wherein the agent is an antibody.

13. The method of claim 9, wherein the agent antagonizes a receptor for TGF-β.

14. The method of claim 9, wherein the agent is a serine/threonine protein kinase inhibitor.

15. The method of claim 9, wherein the agent prevents translocation of a Smad protein from the cytoplasm of the cell to its nucleus.

16. The method of claim 9, wherein the agent is selected from the group consisting of: an extract of amniotic membrane and a purified component of amniotic membrane.

17. The method of claim 9, wherein the agent is a purified component of amniotic membrane selected from the group consisting of: TSG-6, pentraxin (PTX3), thrombospondin, hyaluronic acid (HA), HA-ITI, and lumican.

18. An ex vivo cell culture system comprising a vessel comprising animal cells whose differentiation state is controllable by modulating TGF-β signaling, wherein the animal cells have been expanded by culturing in a medium free of intact amniotic membrane under conditions which downregulate TGF-β signaling in the cells to allow the cells to proliferate without changes to their phenotype.

19. An ex vivo method of preferentially expanding limbal epithelial progenitor cells in a cell culture initiated with a mixture of limbal progenitor cells and transient amplifying cells, the method comprising the steps of:
(a) placing the mixture of limbal epithelial progenitor cells and transient amplifying cells in an ex vivo culture system comprising a culture vessel comprising at least one surface and a medium in contact with the at least one surface, wherein the mixture of cells is seeded in the culture system at a cell density sufficiently low to prevent the transient amplifying cells from having a negative paracrine effect in the limbal epithelial progenitor cells; and
(b) culturing the cells in the ex vivo culture system for a time period exceeding the lifespan of the transient amplifying cells under conditions suitable for expanding the limbal epithelial progenitor cells.

20. The method of claim 20, wherein the cell density is less than about 500 cells/cm² of the at least one surface and the time period is greater than about 3 weeks.

21. An ex vivo method of preferentially expanding limbal epithelial progenitor cells in a cell culture initiated with a mixture of limbal progenitor cells and associated stromal cells wherein at least a portion of the transient amplifying cells have been removed from the mixture of cells, the method comprising the steps of:
(a) placing the mixture of limbal epithelial progenitor cells and associated stromal cells in an ex vivo culture system comprising a culture vessel comprising at least one surface and a medium in contact with the at least one surface, wherein the mixture of cells is seeded in the culture system at a cell density higher than about 10,000 cells/cm² of the at least one surface; and
(b) culturing the cells in the ex vivo culture system.
